(19) United States
(12) Patent Application Publication
(10) Pub. No.: US 2021/0213052 A1
(43) Pub. Date: Jul. 15, 2021

(54) METHOD FOR TREATING ALZHEIMER'S DISEASE

(71) Applicant: NATIONAL TSING HUA UNIVERSITY, Hsinchu (TW)

(72) Inventors: Hsin-Cheng Chiu, Hsinchu (TW); Yuan-Chung Tsai, Hsinchu (TW); Jing-Chian Luo, Hsinchu (TW); Te-I Liu, Hsinchu (TW); Siou-Han Chang, Hsinchu (TW)

(73) Assignee: NATIONAL TSING HUA UNIVERSITY

(21) Appl. No.: 16/996,160

(22) Filed: Aug. 18, 2020

(30) Foreign Application Priority Data
Jan. 13, 2020 (TW) 109101106

(51) Int. Cl. A61K 33/26; A61P 25/28; A61K 9/00

(52) U.S. Cl. CPC A61K 33/26; A61K 9/0009; A61P 25/28

(57) ABSTRACT

The present disclosure provides a method for treating Alzheimer's disease including the following steps. An effective amount of nano-micro magnetic stir bars is administered to a tissue of a subject suffered from Alzheimer's disease. A rotating magnetic field is provided to the subject, wherein each of the nano-micro magnetic stir bars rotates in the tissue correspondingly to the rotating magnetic field. A reacting step is performed for a reaction time, wherein the tissue generates a rotating microflow corresponding to a rotation of each of the nano-micro magnetic stir bars, and a plurality of amyloids are moved within the tissue along with the rotating microflow and then aggregate so as to form a plurality of amyloid aggregates. A removing step is performed, wherein the amyloid aggregates are captured and collected by phagocytes of the tissue so as to be removed from the tissue of the subject.

### RELATED APPLICATIONS

This application claims priority to Taiwan Application Serial Number 109101106, filed Jan. 13, 2020, which is herein incorporated by reference.

### BACKGROUND

### Technical Field

The present disclosure relates to a method for treating Alzheimer's disease.

### Description of Related Art

Generally, amyloid refers to spontaneously formed protein fibers rich in cross-β structure, and the inclusion bodies composed of amyloid are common pathological characteristics of a variety of neurodegenerative diseases.

In the brain tissue of the patient suffered from Alzheimer's disease, β-amyloid (referred to “Aβ”) is found to accumulate outside the necrotic nerve cells. The amyloid-β₄₂ is considered as a biomarker of Alzheimer's disease, and the accumulation of the amyloid-β₄₂ can form amyloid-β₄₂ oligomers (oAβ₄₂) and then lead to severe damage of neuron cells, resulting in cognitive impairment and synaptic dysfunction in the patient suffered from Alzheimer's disease and causing irreversible neurological dysfunction.

Currently, acetylcholinesterase inhibitors are frequently adopted for the clinical treatment of Alzheimer's disease so as to alleviate dementia by slowing down the degrading rate of acetylcholine. However, the treatments of Alzheimer's disease nowadays are still passive methods to delay the disease progression, such as preserving or improving the patient's cognitive function and reducing behavioral disorders and cannot collect and remove the accumulated amyloid.

Therefore, how to develop a system and a method which can collect and remove the accumulate amyloid effectively has become an urgent development goal for those skilled in the art.

### SUMMARY

According to one aspect of the present disclosure, a method for treating Alzheimer's disease includes the following steps. An effective amount of nano-micro magnetic stir bars is administered to a tissue of a subject suffered from Alzheimer's disease, wherein each of the nano-micro magnetic stir bars includes a magnetic material, and an average particle size of the nano-micro magnetic stir bars is 50 nm to 2 μm. A rotating magnetic field is provided to the subject, wherein each of the nano-micro magnetic stir bars rotates in the tissue correspondingly to the rotating magnetic field. A reacting step is performed for a reaction time, wherein the tissue generates a rotating microflow corresponding to a rotation of each of the nano-micro magnetic stir bars, and a plurality of amyloids are moved within the tissue along with the rotating microflow and then aggregate so as to form a plurality of amyloid aggregates. A removing step is performed, wherein the amyloid aggregates are captured and collected by phagocytes of the tissue so as to be removed from the tissue of the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following detailed description of the embodiment, with reference made to the accompanying drawings as follows:

FIG. 1 is a schematic view of a system for collecting amyloid according to one aspect of the present disclosure.

FIG. 2 is a flow chart of a method for operating a system for collecting amyloid according to another aspect of the present disclosure.

FIG. 3 is a flow chart of a method for treating Alzheimer's disease according to further another aspect of the present disclosure.

FIG. 4 shows an intensity analysis result of rotating microflows generated from nano-micro magnetic stir bars corresponding to rotating magnetic fields with different speeds.

FIG. 5 shows images of N2a cells cultured under the rotating magnetic fields with different speeds for 24 hours.

FIG. 6 is a staining result of a sample which is reacted for 20 minutes to remove the amyloid therein.

FIG. 7A shows a result of MTT cell viability assay of N2a cells of a sample.

FIG. 7B shows a result of trypan blue assay of N2a cells of a sample.

FIG. 7C shows a result of lactate dehydrogenase releasing rate of N2a cells of a sample.

FIG. 8 shows a result of a relative amyloid removing index of BV-2 microglia cells.

FIG. 9 shows a result of TNF-α secreted amount of BV-2 microglia cells.

### DETAILED DESCRIPTION

The present disclosure will be further exemplified by the following specific embodiments so as to facilitate utilizing and practicing the present disclosure completely by the people skilled in the art without over-interpreting and over-experimenting. However, these practical details are used to describe how to implement the materials and methods of the present disclosure and are not necessary.

### <System for Collecting Amyloid of the Present Disclosure>

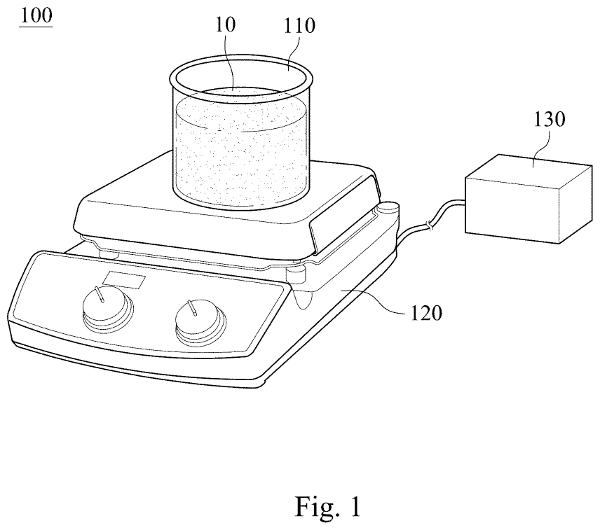

Please refer to FIG. 1, which is a schematic view of a system 100 for collecting amyloid according to one aspect of the present disclosure. The system 100 for collecting amyloid includes a reaction container 110, at least one nano-micro magnetic stir bar (reference number is omitted), a rotating magnetic field supplying device 120 and a driving device 130.

As shown in FIG. 1, a sample 10 is placed in the reaction container 110, and the reaction container 110 includes a plurality of phagocytes (not shown). In detail, the sample 10 can be a tissue including phagocytes and neuron cells or can be a brain tissue, and the nano-micro magnetic stir bar is detachably disposed in the reaction container 110, wherein the nano-micro magnetic stir bar includes a magnetic material. Specifically, in the embodiment of FIG. 1, a number of the nano-micro magnetic stir bar is plural, and the dots in the sample 10 represent the nano-micro magnetic stir bars of the present disclosure. However, it must be noted that the size of the dots and the distribution thereof have no special meanings, and the size and the distribution of the dots do not represent the average particle size of the nano-micro magnetic stir bars or the concentration thereof. Furthermore, the magnetic material of the nano-micro magnetic stir bars can be iron oxide, ferrous oxide, maghemite, ferric tetroxide, or a combination thereof, and an average particle size of the nano-micro magnetic stir bars can be 50 nm to 2 μm.

The rotating magnetic field supplying device 120 is disposed adjacent to the reaction container 110, and the rotating magnetic field supplying device 120 is for applying a rotating magnetic field to the reaction container 110. In detail, the rotating magnetic field supplying device 120 can be a magnetic stirrer or other devices that can provide a rotating magnetic field. Furthermore, the rotating magnetic field supplying device 120 should be disposed adjacent to the reaction container 110 so as to apply the rotating magnetic field to the reaction container 110 and then trigger the nano-micro magnetic stir bars to rotate, but the present disclosure is not limited thereto.

The driving device 130 is electronically connected to the rotating magnetic field supplying device 120, wherein the driving device 130 is for controlling the rotating magnetic field supplying device 120 to operate so as to provide the rotating magnetic field to the reaction container 110. In detail, when the rotating magnetic field supplying device 120 is driven by the driving device 130 and then provides the rotating magnetic field to the reaction container 110, the nano-micro magnetic stir bars will be driven by the rotating magnetic field and rotate in the reaction container 110. As shown in the embodiment of FIG. 1, when the sample 10 is placed in the reaction container 110 and the nano-micro magnetic stir bars rotate in the reaction container 110 simultaneously, the sample 10 will generate a rotating microflow corresponding to the rotations of the nano-micro magnetic stir bars, and a plurality of amyloids (not shown) of the sample 10 will be moved within the sample 10 along with the rotating microflow. In this time, the amyloids moved with the rotating microflow will be captured and collected by the phagocytes. In more detail, the phagocytes will actively approach the amyloids so as to engulf the amyloids and then further remove thereof, and the aforementioned phagocytes can be the cells with the phagocytosis ability, such as microglia, neutrophils, monocytes, macrophages, mast cells, dendritic cells. More preferably, the aforementioned phagocytes can be microglia so as to remove and metabolite the amyloids gently without destroying the sample 10.

Furthermore, the nano-micro magnetic stir bars of the present disclosure can include or can be without the biomolecules, such as peptide fragments, antibodies or glycoproteins, that can target or have an affinity with amyloids, according to actual needs, and the present disclosure is not limited thereto. In detail, when the nano-micro magnetic stir bars of the present disclosure include the peptide fragments, antibodies, glycoproteins or other biomolecules that can target or have the affinity with amyloids, a contacting specificity of the nano-micro magnetic stir bars to the amyloids during the rotating process will be enhanced because of the amyloids targeting property thereof so that the collecting and removing efficiency of the amyloids can be further enhanced. On the contrary, when the nano-micro magnetic stir bars of the present disclosure are without the peptide fragments, antibodies, glycoproteins or other biomolecules that can target or have the affinity with amyloids, the nano-micro magnetic stir bars of the present disclosure also can collide with the amyloids and then are embedded therein during the rotating process so as to form the magnetic amyloid aggregates. Therefore, it is favorable for collecting and removing the amyloids, and the applying field of the system 100 for collecting amyloid of the present disclosure can be more expanded.

Therefore, in the system 100 for collecting amyloid of the present disclosure, by the method that the rotating magnetic field supplying device 120 provides the rotating magnetic field and then the nano-micro magnetic stir bars rotate correspondingly, the amyloids of the sample 10 can be moved within the sample 10 along with the rotating microflow generated from the rotation of the nano-micro magnetic stir bars, and then the amyloids of the sample 10 can be further captured and collected by the phagocytes. Thus, the system 100 for collecting amyloid of the present disclosure has the potentials to remove the amyloids from brain tissue samples and has related market applying potential.

### <Method for Operating a System for Collecting Amyloid of the Present Disclosure>

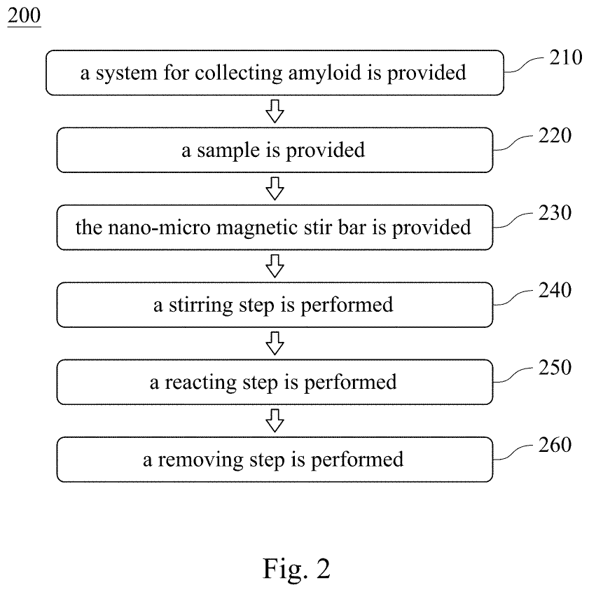

Please refer to FIG. 1 and FIG. 2 simultaneously, wherein FIG. 2 is a flow chart of a method 200 for operating a system for collecting amyloid according to another aspect of the present disclosure. The system 100 for collecting amyloid of FIG. 1 is used to illustrate the details of the method 200 for operating the system for collecting amyloid of the present disclosure. The method 200 for operating the system for collecting amyloid includes Step 210, Step 220, Step 230, Step 240, Step 250 and Step 260.

In Step 210, a system for collecting amyloid is provided. Specifically, the aforementioned system for collecting amyloid can be the system 100 for collecting amyloid of FIG. 1.

In Step 220, a sample 10 is provided, wherein the sample 10 is placed in the reaction container 110, and the sample 10 includes a plurality of amyloids and phagocytes. Specifically, the sample 10 can include neuron cells or can be a brain tissue, and the amyloids can be amyloid-β₄₂ oligomers which have toxicity against the neuron cells, but the present disclosure is not limited thereto.

In Step 230, the nano-micro magnetic stir bar is provided. Specificity, a number of the nano-micro magnetic stir bar can be plural, and a concentration of the nano-micro magnetic stir bars in the sample 10 can be 144 μg/mL to 576 μg/mL. In detail, when the concentration of the nano-micro magnetic stir bars in the sample 10 is too high, the rotating magnetic field may fail to drive each of the nano-micro magnetic stir bars to rotate effectively. Thus, the collecting efficiency of the amyloids will be restricted. Accordingly, in order to drive each of the nano-micro magnetic stir bars to rotate by the rotating magnetic field, the intensity and the speed of the rotating magnetic field must be increased, but the rotating magnetic field with extremely high intensity and extremely high speed may destroy the overall structure of the sample and is unable to gently remove the amyloids therein. On the contrary, when the concentration of the nano-micro magnetic stir bars in the sample 10 is too low, a number and the intensity of the rotating microflows generated from the rotation of the nano-micro magnetic stir bars are insufficient. Thus, the rotating microflows may also fail to drive each of the amyloids to move along therewith, so that the collecting efficiency of the amyloids will be reduced.

In Step 240, a stirring step is performed, wherein the nano-micro magnetic stir bar is placed in the reaction container 110 and the driving device 130 is turned on so as to apply the rotating magnetic field to the reaction container 110. At this time, the nano-micro magnetic stir bar is driven by the rotating magnetic field and rotates in the sample 10, the sample 10 will generate a rotating microflow corresponding to a rotation of the nano-micro magnetic stir bar, and the amyloids of the sample 10 will be moved within the sample 10 along with the rotating microflow. Specifically, a speed of the rotating magnetic field can be 500 rpm to 2500 rpm, and when the aforementioned condition is satisfied, it is favorable for preventing the sample from being destroyed by the rotating magnetic field with extremely high intensity and extremely high speed, and the problem that the speed of the rotating magnetic field being too low to effectively collect amyloid can be further avoided.

In Step 250, a reacting step is performed, wherein the sample 10 is placed in the reaction container 110 and reacts for a reaction time so as to form a plurality of amyloid aggregates by the aggregation of the amyloids. Therefore, it is favorable for collecting and removing the amyloids by the phagocytes. Specifically, the reaction time can be two hours to twenty four hours so as to collect and remove the amyloids effectively.

In Step 260, a removing step is performed, wherein the amyloids moved within the sample 10 along with the rotating microflow are captured and collected by the phagocytes so as to remove the amyloids from the sample 10.

Furthermore, a number of the nano-micro magnetic stir bar can be plural, the amyloid aggregates can include a plurality of magnetic amyloid aggregates, and each of the magnetic amyloid aggregates is coupled with at least one of the nano-micro magnetic stir bars. Specifically, when the amyloids are moved within the sample 10 along with the rotating microflows generated from the rotation of each of the nano-micro magnetic stir bars, the amyloids which form as an aggregate will have the opportunity to contact with the nano-micro magnetic stir bars being rotating, and then the nano-micro magnetic stir bars may be embedded therein and coupled therewith so as to form the magnetic amyloid aggregates. Moreover, because the magnetic amyloid aggregates include the nano-micro magnetic stir bars, the magnetic amyloid aggregates have a magnetic attraction, and the magnetic amyloid aggregates will attract each other to form a larger magnetic amyloid aggregate. Therefore, it is favorable for the magnetic amyloid aggregates to be captured and collected by the phagocytes, so that the collecting efficiency of the amyloids can be further enhanced. Furthermore, because the magnetic amyloid aggregates have larger sizes, the phagocytes will actively approach and engulf the magnetic amyloid aggregates with larger sizes and then further remove the amyloids thereof.

Therefore, the method 200 for operating the system 100 for collecting amyloid of the present application can effectively capture and remove the amyloids moved along with the rotating microflow generated from the nano-micro magnetic stir bar by the phagocytes, so that the method 200 for operating the system 100 for collecting amyloid of the present application has the potentials to remove the amyloids from brain tissue samples and has related market applying potential.

### <Method for Treating Alzheimer's Disease of the Present Disclosure>

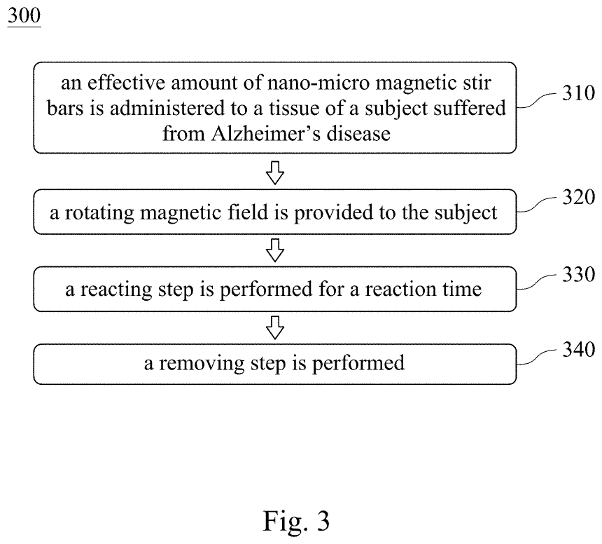

Please refer to FIG. 3, which is a flow chart of a method 300 for treating Alzheimer's disease according to further another aspect of the present disclosure. The method 300 for treating Alzheimer's disease of the present disclosure includes Step 310, Step 320, Step 330 and Step 340.

In Step 310, an effective amount of nano-micro magnetic stir bars is administered to a tissue of a subject suffered from Alzheimer's disease, wherein each of the nano-micro magnetic stir bars includes a magnetic material, and an average particle size of the nano-micro magnetic stir bars is 50 nm to 2 μm. Specificity, the magnetic material of the nano-micro magnetic stir bars can be iron oxide, ferrous oxide, maghemite, ferric tetroxide, or a combination thereof, and the effective amount of the nano-micro magnetic stir bars can be 144 μg/mL to 576 μg/m L. In detail, when the concentration of the nano-micro magnetic stir bars in the tissue is too high, the rotating magnetic field may fail to drive each of the nano-micro magnetic stir bars to rotate effectively. Accordingly, in order to drive each of the nano-micro magnetic stir bars to rotate by the rotating magnetic field, the intensity and the speed of the rotating magnetic field must be increased, but the rotating magnetic field with extremely high intensity and extremely high speed may destroy the overall structure of the tissue and is unable to gently remove the amyloids therein. On the contrary, when the concentration of the nano-micro magnetic stir bars in the tissue is too low, a number and an intensity of the rotating microflows generated from the rotation of the nano-micro magnetic stir bar are insufficient. Thus, the rotating microflows may also fail to drive each of the amyloids to move along therewith, so that the collecting efficiency of the amyloids will be reduced. Furthermore, the tissue of the subject can include a neuron cell or can be a brain tissue.

In Step 320, a rotating magnetic field is provided to the subject, wherein each of the nano-micro magnetic stir bars rotates in the tissue correspondingly to the rotating magnetic field. Specifically, a speed of the rotating magnetic field is 500 rpm to 2500 rpm, and when the aforementioned condition is satisfied, it is favorable for preventing the tissue from being destroyed by the rotating magnetic field with extremely high intensity and extremely high speed, and the situation that the speed of the rotating magnetic field being too low to effectively collect amyloid can be further avoided.

In Step 330, a reacting step is performed for a reaction time, wherein the tissue generates a rotating microflow corresponding to a rotation of each of the nano-micro magnetic stir bars, and a plurality of amyloids are moved within the tissue along with the rotating microflow and then aggregate so as to form a plurality of amyloid aggregates. In detail, the reaction time is two hours to twenty four hours so as to collect and remove the amyloids effectively. Furthermore, the amyloids can be amyloid oligomers, the amyloid aggregates can include a plurality of magnetic amyloid aggregates, and each of the magnetic amyloid aggregates is coupled with at least one of the nano-micro magnetic stir bars. Specifically, when the amyloids are moved within the tissue along with the rotating microflows generated from the rotation of each of the nano-micro magnetic stir bars, the amyloids which form as an aggregate will have the opportunity to contact with the nano-micro magnetic stir bar being rotating, and then the nano-micro magnetic stir bars may be embedded therein and coupled therewith so as to form the magnetic amyloid aggregates. Moreover, because the magnetic amyloid aggregates include the nano-micro magnetic stir bars, the magnetic amyloid aggregates have a magnetic attraction, and the magnetic amyloid aggregates will attract each other to form a larger magnetic amyloid aggregate. Therefore, it is favorable for the magnetic amyloid aggregates to be captured and collected by the phagocytes, so that the collecting efficiency of the amyloids can be further enhanced. Furthermore, because the magnetic amyloid aggregates have larger sizes, the phagocytes will actively approach and engulf the magnetic amyloid aggregates with larger sizes and then further remove the amyloids thereof.

In Step 340, a removing step is performed, wherein the amyloid aggregates are captured and collected by phagocytes of the tissue so as to be removed from the tissue of the subject. In detail, the tissue of the subject includes phagocytes, and the phagocytes can be the cells with the phagocytosis ability, such as microglia, neutrophils, monocytes, macrophages, mast cells, dendritic cells. More preferably, the aforementioned phagocytes can be microglia so as to remove and metabolite the amyloids gently without destroying the sample.

Therefore, the method 300 for treating Alzheimer's disease of the present disclosure can effectively capture and remove the amyloids moved along with the rotating microflow generated from the nano-micro magnetic stir bar by the phagocytes, so that the method 300 for treating Alzheimer's disease of the present disclosure has the potentials to remove the amyloids from brain tissue, to treat Alzheimer's disease and has related market applying potential.

### Example and Comparative Example

The present disclosure will be further exemplified by the following specific examples so as to describe the biocompatibility, the amyloids collecting efficiency and amyloids removing efficiency of the system for collecting amyloid, the method for operating a system for collecting amyloid and the method for treating Alzheimer's disease of the present disclosure. Furthermore, the system for collecting amyloid and the method for operating a system for collecting amyloid described as foregoing aspects will be used to illustrate the following examples, so that the details thereof will not be described again hereafter.

### I. Intensity Analysis of the Rotating Microflows Generated from the Rotation of the Nano-Micro Magnetic Stir Bars

The intensity analysis of the rotating microflows generated from the rotation of the nano-micro magnetic stir bars is performed by Example 1, Example 2, Example 3 and Comparative example 1. In detail, all of the samples of Example 1, Example 2, Example 3 and Comparative example 1 are PBS solution including 144 μg/mL of the nano-micro magnetic stir bars. In the present experiment, an equal amount of Rhodamine B reagent is added in each of the samples of Example 1, Example 2, Example 3 and Comparative example 1, and the rotating magnetic fields with different speeds are respectively applied thereto and react for 0 second (that is, the rotating magnetic field is not applied thereto), 1 second, 5 seconds and 10 seconds so as to observe the dispersed situation of the Rhodamine B reagent in the PBS solution caused by the rotating microflows generated from the rotation of the nano-micro magnetic stir bars in the sample. The speeds of the rotating magnetic fields are 500 rpm in Example 1, 1500 rpm in Example 2 and 2500 rpm in Example 3, and the Comparative example 1 is without any rotating magnetic field applied thereto so as to analyze the intensity of the rotating microflows generated from the rotation of the nano-micro magnetic stir bars of the present disclosure.

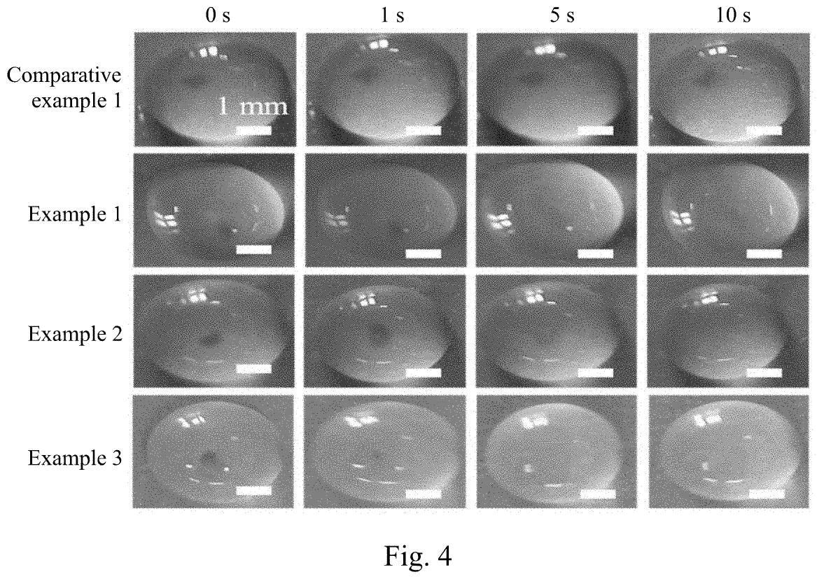

Please refer to FIG. 4, which shows an intensity analysis result of the rotating microflows generated from the nano-micro magnetic stir bars corresponding to the rotating magnetic fields with different speeds. As shown in FIG. 4, the Rhodamine B reagent in Example 1, Example 2 and Example 3 is gradually diffused into the sample along with the increase of the reaction time after applying the rotating magnetic field thereto, wherein the diffusing efficiency of Example 3 is the best under the speed of the rotating magnetic field being 2500 rpm. On the contrary, under the condition that Comparative example 1 is without any rotating magnetic field applied thereto, the Rhodamine B reagent cannot be diffused into the sample and remains in the same distribution position and shape therein. Therefore, as shown in the aforementioned results, when the nano-micro magnetic stir bars of the present disclosure are driven to rotate by the rotating magnetic field with a speed of 500 rpm to 2500 rpm, the sample can generate a rotating microflow with a proper intensity. Thus, the amyloids of the sample can be moved within the sample along with the rotating microflow, making it have relevant applying potentials.

### II. Biocompatibility Test

The biocompatibility test of the present disclosure is performed by using the PBS solution including 144 μg/mL of the nano-micro magnetic stir bars of the aforementioned Example 1, Example 2 and Example 3 to treat 3×10⁵ of Neuro-2a cells (“N2a cells” hereafter) so as to observe the cell viability of the N2a cells in the sample after treating with the rotating microflow generated correspondingly to the rotating magnetic fields with a speed of 500 rpm to 2500 rpm and then to estimate the biocompatibility of the system for collecting amyloid, the method for operating a system for collecting amyloid and the method for treating Alzheimer's disease of the present disclosure. Furthermore, the present experiment further includes Control group and the aforementioned Comparative example 1, wherein the sample of Control group is a PBS solution without any nano-micro magnetic stir bar and without any rotating magnetic field applied thereto so as to further illustrate the biocompatibility of the nano-micro magnetic stir bars of the present disclosure to the N2a cells.

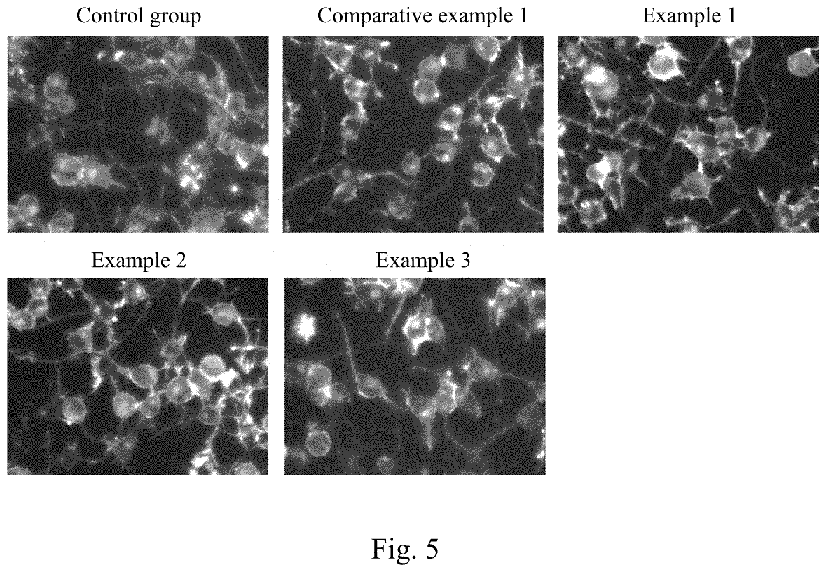

Please refer to FIG. 5, which shows images of N2a cells cultured under the rotating magnetic fields with different speeds for 24 hours. As shown in FIG. 5, under the condition that the sample does not include the nano-micro magnetic stir bars and is without the rotating magnetic field applied thereto, the N2a cells of Control group have integral and elongated axons, and under the condition that the sample includes the nano-micro magnetic stir bars but is without the rotating magnetic field applied thereto, the N2a cells of Comparative example 1 also have integral and elongated axons after reacting for 24 hours. Accordingly, it is shown that the N2a cells cultured with the nano-micro magnetic stir bars of the present invention will not inhibit the growth and activity thereof. Furthermore, in Example 1, Example 2 and Example 3, after reacting for 24 hours under the rotating magnetic field with a speed being 500 rpm to 2500 rpm, the N2a cells of all of Example 1, Example 2 and Example 3 have integral and elongated axons. Moreover, under the condition that the cell viability of the N2a cells of Control group is 100%, the cell viability of the N2a cells of Example 1 is about 96%, the cell viability of the N2a cells of Example 2 is 94%, and the cell viability of the N2a cells of Example 3 is 95%. Therefore, it is shown that the biocompatibility of the system for collecting amyloid, the method for operating a system for collecting amyloid and the method for treating Alzheimer's disease of the present disclosure is excellent, and the system for collecting amyloid, the method for operating a system for collecting amyloid and the method for treating Alzheimer's disease of the present disclosure can remove the amyloids gently from the sample without destroying thereof. Thus, the system for collecting amyloid, the method for operating a system for collecting amyloid and the method for treating Alzheimer's disease of the present disclosure have potentials to remove the amyloids from brain tissue samples, to treat Alzheimer's disease and has related market applying potential.

### III. Amyloids Collecting Rate Analysis

The amyloids collecting rate analysis is performed by Example 4, Example 5 and Example 6. In detail, the samples of all of Example 4, Example 5 and Example 6 are PBS solution including 20 μM of amyloid oligomers and a rotating magnetic field with a speed of 2500 rpm is applied thereto, wherein the concentration of the nano-micro magnetic stir bars of Example 4 is 144 μg/mL, the concentration of the nano-micro magnetic stir bars of Example 5 is 288 μg/mL, and the concentration of the nano-micro magnetic stir bars of Example 6 is 576 μg/mL.

Furthermore, the present experiment further includes Comparative example 2 and Comparative example 3, wherein the samples of both of Comparative example 2 and Comparative example 3 are PBS solution including 20 μM of amyloid oligomers and without any rotating magnetic field applied thereto, and Comparative example 3 further includes 576 μg/mL of the nano-micro magnetic stir bars compared to Comparative example 2 so as to estimate the amyloids collecting rate of the system for collecting amyloid, the method for operating a system for collecting amyloid and the method for treating Alzheimer's disease of the present disclosure.

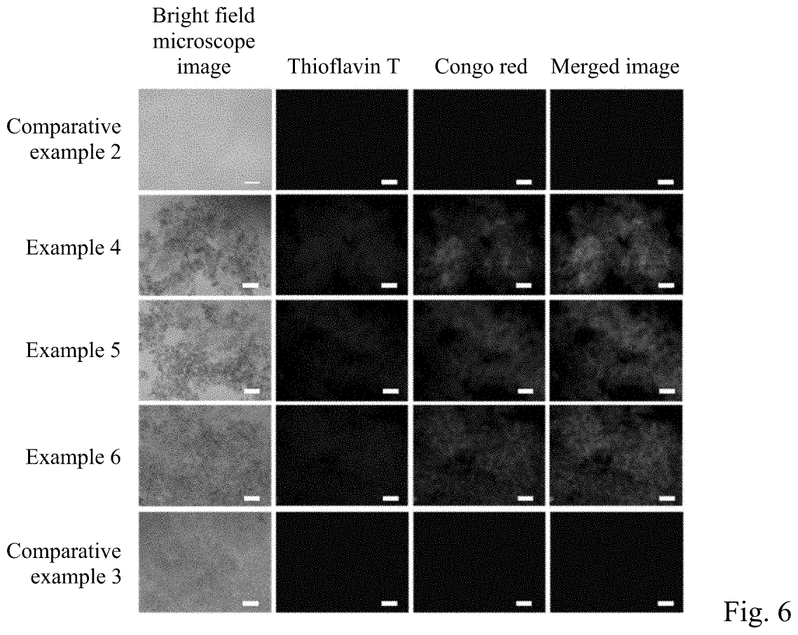

Please refer to FIG. 6, which is a staining result of a sample which is reacted for 20 minutes to remove the amyloid therein. In detail, after reacting for 20 minutes, the amyloids in the samples of Example 4, Example 5 and Example 6 will form as amyloid aggregates, and the amyloid aggregates are coupled with the nano-micro magnetic stir bars and then form as magnetic amyloid aggregates. Specificity, the magnetic amyloid aggregates can be stained by Thioflavin T and Congo red, and the magnetic amyloid aggregates will attract each other to form a larger magnetic amyloid aggregate, so that the intensity of the fluorescence signals of Thioflavin T and Congo red thereof will be further enhanced. As shown in FIG. 6, each of the bright field microscope images of Example 4, Example 5 and Example 6 after reacting for 20 minutes shows a aggregation of the amyloid aggregates, and all of the fluorescence signals of Thioflavin T and Congo red of Example 4, Example 5 and Example 6 are significantly increased compared to that of Comparative example 2 and Comparative example 3. Accordingly, it is shown that the amyloids do couple with the nano-micro magnetic stir bars and form large magnetic amyloid aggregates that are easy to remove and can be engulfed by the phagocytes.

As shown in the aforementioned results, the system for collecting amyloid, the method for operating a system for collecting amyloid and the method for treating Alzheimer's disease of the present disclosure can make the amyloids of the sample aggregate and form as aggregates, so that the system for collecting amyloid, the method for operating a system for collecting amyloid and the method for treating Alzheimer's disease of the present disclosure have potentials to remove the amyloids from brain tissue samples, to treat Alzheimer's disease and has related market applying potential.

### IV. Cell Viability Analysis

Example 7 is used to perform MTT cell viability assay, Trypan blue assay and the lactate dehydrogenase (LDH) releasing analysis so as to estimate the cell viability of the cells of tissue treated by the system for collecting amyloid, the method for operating a system for collecting amyloid and the method for treating Alzheimer's disease of the present disclosure. In detail, the sample of Example 7 includes a cell culture medium including 1.2×10⁴ of N2a cells, wherein the concentration of the amyloid oligomers of the cell culture medium of Example 7 is 160 μM, a concentration of the nano-micro magnetic stir bars thereof is 144 μg/mL, and a rotating magnetic field with a speed of 2500 rpm is applied thereto so as to estimate the cell viability of the N2a cells treated by the system for collecting amyloid, the method for operating a system for collecting amyloid and the method for treating Alzheimer's disease of the present disclosure used to collecting and removing the amyloids.

In the MTT cell viability assay, the sample of Example 7 is incubated in an incubator at 37° C., 5% CO₂ for 24 hours, and then the cultured medium of the sample is removed and 100 μL of 0.25 mg/mL MTT reagent is added therein and reacted at 37° C. for 4 hours. After reacting for 4 hours, the MTT reagent is removed and 200 μL of dimethyl sulfoxide (DMSO) is added to the sample so as to dissolve the crystals generated from the reaction between the MTT reagent and the N2a cells, and then an absorbance at 570 nm thereof is measured by a spectrophotometer and further converted into the data of the cell viability of the N2a cells.

In the Trypan blue assay, the sample of Example 7 is incubated in an incubator at 37° C., 5% CO2 for 24 hours, and then the cultured medium of the sample is removed and 4% Trypan blue solution is added therein and reacted at the room temperature for 5 minutes. After reacting for 5 minutes, the sample is observed by a microscope so as to calculate the numbers of living cells and death cells, and the numbers of living cells and death cells are further converted into the data of the cell viability of the N2a cells.

In the lactate dehydrogenase releasing analysis, the sample of Example 7 is incubated in an incubator at 37° C., 5% CO₂ for 24 hours, and then the cultured medium of the sample is removed and 50 μL of the lactate dehydrogenase detecting reagent (Catalog No. AC211760050, Thermo Fisher Scientific) is added therein and reacted at 37° C. for 2 hours. After reacting for 2 hours, an absorbance at 450 nm of the sample is measured by a spectrophotometer. In detail, the lactate dehydrogenase is an enzyme within the cells, so when a cell is damaged, the lactate dehydrogenase thereof will release from the cell. Accordingly, the greater amount of the lactate dehydrogenase is released, the higher of the value of the absorbance at 450 nm is, so that the data of the value of the absorbance at 450 nm will be further converted into the data of the cell viability of the N2a cells.

Furthermore, the present experiment further includes the aforementioned Control group and Comparison Group 1, Comparison Group 2, Comparative example 4 and Comparative example 5, wherein all of the samples of Comparison Group 1, Comparison Group 2, Comparative example 4 and Comparative example 5 include a cell culture medium including 1.2×10⁴ of N2a cells. In detail, both of the samples of Comparison Group 1 and Comparison Group 2 include 144 μg/mL of the nano-micro magnetic stir bars but are without any amyloid oligomer, and the sample of Comparison Group 2, compared to Comparison Group 1, further includes a rotating magnetic field with a speed of 2500 rpm applied thereto. Furthermore, both of the samples of Comparative example 4 and Comparative example 5 include 160 μM of amyloid oligomers but are without any rotating magnetic field applied thereto, and the sample of Comparative example 5, compared to Comparative example 4, further includes 144 μg/mL of the nano-micro magnetic stir bars so as to estimate the cell viability of the N2a cells treated by the system for collecting amyloid, the method for operating a system for collecting amyloid and the method for treating Alzheimer's disease of the present disclosure used to collecting and removing the amyloids.

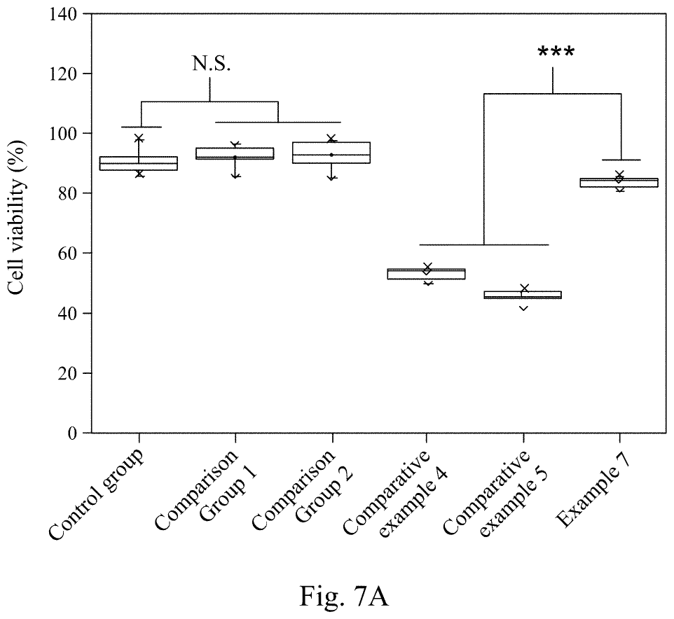

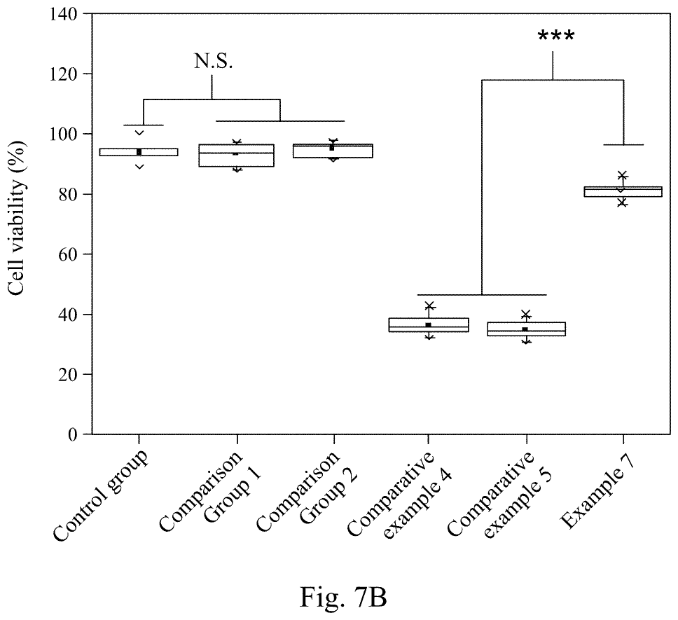

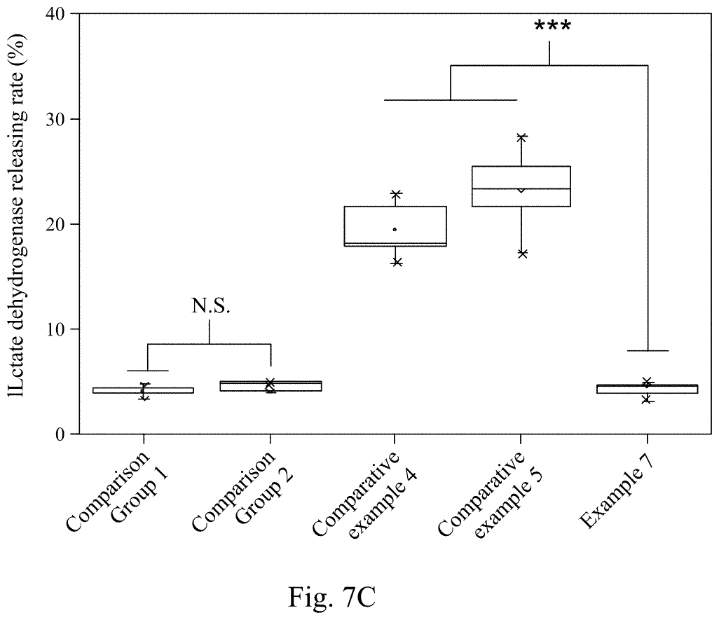

Please refer to FIG. 7A, FIG. 7B and FIG. 7C, wherein FIG. 7A shows a result of MTT cell viability assay of N2a cells of a sample, FIG. 7B shows a result of trypan blue assay of N2a cells of a sample, and FIG. 7C shows a result of lactate dehydrogenase releasing rate of N2a cells of a sample.

As shown in FIG. 7A and FIG. 7B, the results of the MTT cell viability assay and Trypan blue assay of Comparison Group 1, Comparison Group 2 and Example 7 are similar with that of Control group, wherein under the condition of Comparison Group 2 which has the rotating magnetic field of 2500 rpm applied thereto and includes 144 μg/mL of the nano-micro magnetic stir bars, the N2a cells will not be damaged. Thus, the survive of N2a cells will not be affected when the N2a cells are cultured with the nano-micro magnetic stir bars of the present disclosure, and the nano-micro magnetic stir bars of the present disclosure has an excellent biocompatibility. Furthermore, the cell viability of Example 7 is about 84% in the MTT cell viability assay and is about 82% in the Trypan blue assay, which is significantly higher than that of Comparative example 4 and Comparative example 5.

As shown in FIG. 7C, the lactate dehydrogenase releasing rate of the N2a cells of Example 7 is about 4%, which is similar with that of Comparison Group 1 and Comparison Group 2 and is significantly lower than the lactate dehydrogenase releasing rate of Comparative example 4 being 20% and the lactate dehydrogenase releasing rate of Comparative example 5 being 23%. Therefore, it is shown that the system for collecting amyloid, the method for operating a system for collecting amyloid and the method for treating Alzheimer's disease of the present disclosure can effectively capture the amyloids and prevent the amyloids from entering the neuron cells and being toxicity against the neuron cells. Thus, the system for collecting amyloid, the method for operating a system for collecting amyloid and the method for treating Alzheimer's disease of the present disclosure have potentials to remove the amyloids from brain tissue samples, to treat Alzheimer's disease and has related market applying potential.

### V. The Amyloids Removing Rate Analysis

The amyloids removing rate analysis of the present disclosure is performed by Example 8, Example 9 and Example 10 including BV-2 microglial cells so as to estimate the ability of BV-2 microglial cells to remove different types of amyloids and estimate its effect on the secretion of TNF-a. In detail, the amyloids of Example 8 are amyloid oligomers, the amyloids of Example 9 are amyloids aggregates without the nano-micro magnetic stir bars, and the amyloids of Example 10 are magnetic amyloid aggregates including the nano-micro magnetic stir bars coupled therein.

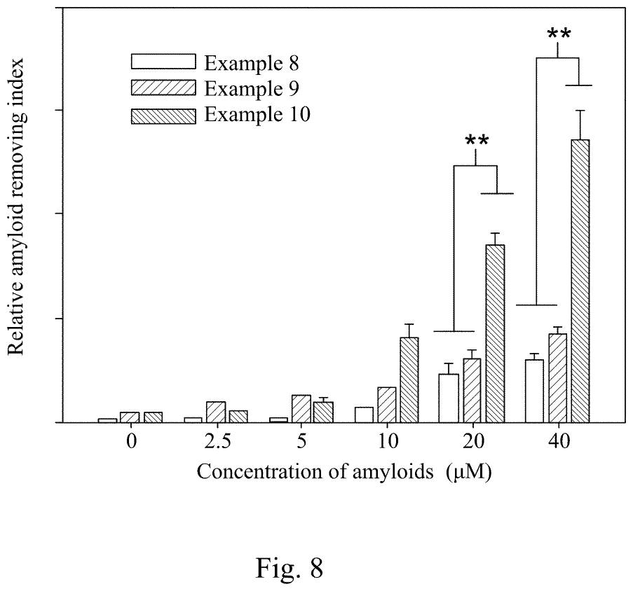

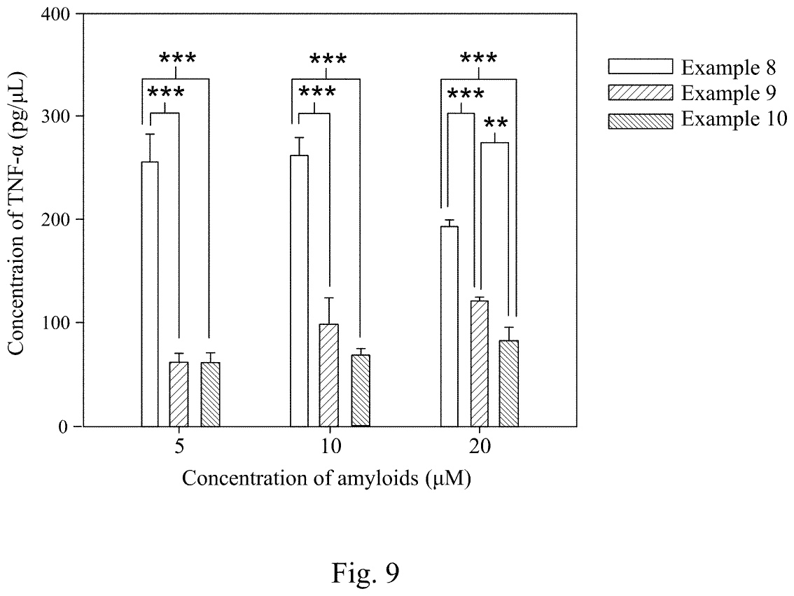

Please refer to FIG. 8 and FIG. 9, wherein FIG. 8 shows a result of a relative amyloid removing index of BV-2 microglia cells, and FIG. 9 shows a result of TNF-α secreted amount of BV-2 microglia cells.

As shown in FIG. 8, the relative amyloid removing indexes of Example 8, Example 9 and Example 10 are increased along with the increase of the concentration of the amyloids, wherein the relative amyloid removing index of Example 10 is the highest, which is 15% at the concentration of the amyloids being 40 μM. Accordingly, the system for collecting amyloid, the method for operating a system for collecting amyloid and the method for treating Alzheimer's disease of the present disclosure can effectively collect and remove different types of amyloids, and the removing efficiency to the magnetic amyloid aggregates is the best. Furthermore, as shown in FIG. 9, the TNF-α secreted amount of BV-2 microglia cells of Example 10 is the lowest, and the TNF-α secreted amount of BV-2 microglia cells of Example 9 is the second. Therefore, it is shown that the system for collecting amyloid, the method for operating a system for collecting amyloid and the method for treating Alzheimer's disease of the present disclosure can effectively remove different types of amyloids, and the magnetic amyloid aggregates can further effectively drive the BV-2 microglial cells to reduce the release of TNF-α so as to slow the damage to neuron cells in the sample.

To sum up, by the method that the rotating magnetic field supplying device provides the rotating magnetic field and then drive the nano-micro magnetic stir bars to rotate, the amyloids of the sample can be moved within the sample along with the rotating microflow generated from the rotation of the nano-micro magnetic stir bars and then are captured and collected by the phagocytes so as to remove and metabolite the amyloids gently without destroying the sample. Therefore, the system for collecting amyloid, the method for operating a system for collecting amyloid and the method for treating Alzheimer's disease of the present disclosure have potentials to remove the amyloids from brain tissue samples, to treat Alzheimer's disease and has related market applying potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A method for treating Alzheimer's disease, comprising:
administering an effective amount of nano-micro magnetic stir bars to a tissue of a subject suffered from Alzheimer's disease, wherein each of the nano-micro magnetic stir bars comprises a magnetic material, and an average particle size of the nano-micro magnetic stir bars is 50 nm to 2 μm;
providing a rotating magnetic field to the subject, wherein each of the nano-micro magnetic stir bars rotates in the tissue correspondingly to the rotating magnetic field;
performing a reacting step for a reaction time, wherein the tissue generates a rotating microflow corresponding to a rotation of each of the nano-micro magnetic stir bars, and a plurality of amyloids are moved within the tissue along with the rotating microflow and then aggregate so as to form a plurality of amyloid aggregates; and
performing a removing step, wherein the amyloid aggregates are captured and collected by phagocytes of the tissue so as to be removed from the tissue of the subject.

2. The method of claim 1, wherein the magnetic material is iron oxide, ferrous oxide, maghemite, ferric tetroxide, or a combination thereof.

3. The method of claim 1, wherein the reaction time is two hours to twenty four hours.

4. The method of claim 3, wherein the amyloid aggregates include a plurality of magnetic amyloid aggregates, and each of the magnetic amyloid aggregates is coupled with at least one of the nano-micro magnetic stir bars.

5. The method of claim 1, wherein a speed of the rotating magnetic field is 500 rpm to 2500 rpm.

6. The method of claim 1, wherein the effective amount of the nano-micro magnetic stir bars is 144 μg/mL to 576 μg/mL.

7. The method of claim 1, wherein the amyloids are amyloid oligomers.

8. The method of claim 1, wherein the tissue of the subject comprises a neuron cell.

9. The method of claim 8, wherein the tissue of the subject is a brain tissue.

10. The method of claim 1, wherein the phagocytes are microglia.
